(19) United States
(12) Patent Application Publication
(10) Pub. No.: US 2024/0065975 A1
(43) Pub. Date: Feb. 29, 2024

(54) OTF COMPOUND CONNECTED BY SEWING

(71) Applicant: LTS LOHMANN THERAPIE-SYSTEME AG, Andernach (DE)

(72) Inventors: Michael LINN, Waldböckelheim (DE); Mario FICKER, Bonn (DE); Christoph SCHMITZ, Rheinbrohl (DE); Markus MÜLLER, Troisdorf (DE)

(21) Appl. No.: 18/272,405

(22) PCT Filed: Jan. 14, 2022
(86) PCT No.: PCT/EP2022/050798

§ 371 Date: Jul. 14, 2023

(30) Foreign Application Priority Data
Jan. 15, 2021 (DE) 10 1021 100 782.8

(51) Int. Cl. A61K 9/00; A61K 31/135; A61K 47/26

(52) U.S. Cl. CPC A61K 9/006; A61K 31/135; A61K 47/26

(57) ABSTRACT

The present invention relates to a multi-layer thin film comprising at least two layers arranged one on top of the other which each comprise at least one polymer, these at least two layers being connected to one another by at least one stitched seam using at least one thread, and to a method for producing such a thin film and to the use thereof as a medicament.

The present invention relates to a multi-layer thin film comprising at least two layers arranged one on top of the other which each comprise at least one polymer, these at least two layers being connected to one another by at least one stitched seam using at least one thread, and to a method for producing such a thin film and to the use thereof as a medicament.

Oral thin films are thin films containing an active pharmaceutical ingredient that are placed directly in the oral cavity or against the oral mucosa and dissolve there. These films are, especially, thin active-ingredient-containing polymer-based films which, when applied to a mucous membrane, especially the oral mucosa, deliver the active ingredient directly into same. These oral thin films are not generally sticky outwardly. The very good blood supply to the oral mucosa ensures a rapid transfer of the active ingredient into the bloodstream. This dosage system has the advantage that the active ingredient is absorbed for the most part by the mucous membrane, thus avoiding the first-pass effect, which occurs in the case of the conventional dosage form of an active ingredient in tablet form, which generally has to be taken with liquid, which may be disadvantageous. The active ingredient may be dissolved, emulsified or dispersed in the film. Suitable active ingredients may also be swallowed once the oral thin film has dissolved in the mouth, and thus may be absorbed via the gastrointestinal tract.

The oral thin films known from the prior art have the disadvantage that the maximum area density and thus the amount of contained active pharmaceutical ingredient is determined, amongst other things, by the drying of the oral thin film during production thereof. The greater is the area density of the oral thin film, the greater the amount of active pharmaceutical ingredient that may be contained therein, however, the drying time of the oral thin film is extended, as a result, to a time that is no longer economical. This disadvantage may indeed be counteracted by an increased drying temperature, however, the active pharmaceutical ingredient is thus exposed to an undesirable thermal load. In addition, oral thin films with a high area density have a relatively long breakdown time, which may be undesirable depending on the application.

The above-mentioned problems can be overcome in principle by multi-layer oral thin films.

Multi-layer oral thin films are known from the prior art.

For example, WO 2011/134846 A1 discloses multi-layer oral thin films comprising an active-ingredient-containing layer and a layer containing a substance that is incompatible with the active ingredient in the active-ingredient-containing layer, these two layers being separated by a further protective layer disposed between these two layers.

US 2013/0017235 A1 discloses multi-layer oral thin films in which an active-ingredient-containing layer is enclosed by two water-swellable polymer layers.

The known multi-layer oral thin films, however, in which a plurality of individual layers with a relatively low area density are adhesively bonded or laminated to form a compound, have a number of disadvantages. On the one hand, greater amounts of suitable adhesives have to be used, as appropriate, between the layers. On the other hand, such multi-layer systems can be produced by firstly producing a first layer and, once this has dried, laminating a second layer onto the first layer. Once the second layer has dried on the first layer, a third layer can then be laminated on as appropriate. By means of such a method, multi-layer thin films can indeed be provided, but only in each case by laminating further layers onto an existing layer in each case. This, in turn, has the disadvantage that the active pharmaceutical ingredient is more strongly thermally loaded on account of multiple coating processes. In addition, a compound created by lamination, especially if the individual layers of the oral thin film are not sticky, is often unstable and may break down easily.

Furthermore, such multi-layer oral thin films have the disadvantage that a solid flat compound is created, so that material exchange or migration may occur between the layers. Especially, the migration of auxiliary substances or of an active pharmaceutical ingredient from one layer into the other may be problematic.

The aim of the present invention lies in overcoming the above-mentioned disadvantages of the prior art. Especially, the aim of the present invention lies in providing a multi-layer oral thin film that is constructed from a plurality of individual layers that are fixedly connected to one another. The individual layers, however, should have such an area density that the individual layers can be dried in an economically acceptable time and the active pharmaceutical ingredient is thermally loaded to a lesser extent. Furthermore, such a migration or material exchange between the individual layers is to be prevented wherever possible. The multi-layer oral thin film should, additionally, dissolve relatively quickly in the case of application in the oral cavity. In addition, it is desirable if individual layers of the multi-layer oral thin film can be provided with markings.

The above aim is addressed by a multi-layer oral thin film according to claim 1, the multi-layer oral thin film comprising at least two layers arranged one on top of the other, which each comprise at least one polymer, these at least two layers being connected to one another by at least one stitched seam using at least one thread.

Such a multi-layer oral thin film is distinguished in that, by using a plurality of layers, the amount of active pharmaceutical ingredient, in relation to the total oral thin film, can be increased without high area densities and associated long drying times being necessary or without having to accept a thermal loading of the active pharmaceutical ingredient. Furthermore, since the at least two layers are only sewn together once dry, it can be largely ensured that there is no migration or material exchange between the individual layers.

Especially, due to the fact that the stitching not provided over the entire surface area, the saliva can penetrate between the individual layers and can thus cause the oral thin film to dissolve more quickly.

In addition, the use of a thread that has a different colour than at least one of the at least two layers allows for a marking of the multi-layer oral thin film.

In the present document, the word “comprising” can also mean “consisting of”.

The term “sewing” means, especially, the connection of materials by a stitched seam. Especially, substantially two-dimensional materials can be connected to one another by sewing. The sewing is preferably defined as the process in which one or more threads are guided, especially repeatedly many times over, through the material to be sewn, the threads being looped during the process with one another or with the material to be sewn. A definition of “sewing” can be found for example in DIN61400 (2002).

A thread preferably comprises a structure that has a dominant one-dimensional extent and a uniformity in the longitudinal direction. The thread can be (theoretically) endless (for example thread on a reel) or length-limited (for example thread in a sewing needle).

The multi-layer oral thin film according to the invention is preferably characterised in that the at least one polymer comprises at least one water-soluble polymer.

Water-soluble polymers comprise chemically very different natural or synthetic polymers, the common feature of which is their solubility in water or aqueous media. A precondition is that these polymers have a number of hydrophilic groups sufficient for the water solubility and are not crosslinked. The hydrophilic groups may be non-ionic, anionic, cationic and/or zwitterionic.

Water-soluble is preferably understood to mean a solubility in water of greater than 100 g/L at 25° C.

The at least one water-soluble polymer is preferably selected from the group comprising starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, vinyl pyrrolidone/vinyl acetate copolymers, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums, with polyvinyl alcohols and copolymers thereof being especially preferred.

The at least two layers of the multi-layer oral thin film may each comprise the same polymer.

In another embodiment the at least two layers of the multi-layer oral thin film may each comprise a different polymer.

Furthermore, embodiments are conceivable in which one layer of the multi-layer oral thin film forms a backing layer, which preferably does not contain an active pharmaceutical ingredient. The term “backing layer” is understood to mean a layer of the multi-layer oral thin film that is one of the outermost layers of the multi-layer oral thin film. The active pharmaceutical ingredient is preferably contained only in the at least one, other layer, which is not the backing layer.

A backing layer can prevent liquid from penetrating the formulation and dissolving it too quickly, so that the active ingredient remains at the application site for the maximum time to achieve the greatest possible permeation through the mucosa or a delayed release. Another effect of the backing layer is that it prevents the administered film from detaching from the application site and adhering elsewhere, such as the teeth. Furthermore, the bad taste caused by the active ingredient can be concealed by a backing layer. The active ingredient can also be protected from additional saliva, which is advantageous especially if the active ingredient or the active ingredient permeation is pH-sensitive and the pH value would change as a result of too much saliva.

Insoluble or slowly soluble polymers, such as polyethylene glycols, or polymer films are often used as material for such backing layers. These have the disadvantage, however, that they have to be removed or swallowed following the application.

The multi-layer oral thin film according to the invention is preferably characterised in that the at least one polymer, preferably the water-soluble polymer, is provided in the relevant layer of the multi-layer oral thin film in an amount of from 10 to 90 wt. %, preferably from 20 to 60 wt. %, especially preferably from 30 to 50 wt. %, in relation to the total weight of the layer in question.

The multi-layer oral thin film according to the invention is further preferably characterised in that at least one of the layers comprises at least one active pharmaceutical ingredient.

The at least one active pharmaceutical ingredient is in principle not subject to any restriction, but is preferably selected from all active pharmaceutical ingredients which are suitable for oral and/or transmucosal application.

According to the present invention, all pharmaceutically acceptable salts and solvates of the particular active pharmaceutical ingredient are also subsumed under the active pharmaceutical ingredient.

Preferred active ingredients are selected from the group comprising the active ingredient classes of analgesics, hormones, hypnotics, sedatives, antiepiletics, analeptics, psychoneurotropic drugs, neuro-muscle blockers, antspasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active ingredients, antibiotics, chemotherapeutics and/or narcotics.

The at least one active pharmaceutical ingredient is especially preferably ketamine and/or a pharmaceutically acceptable salt or solvate thereof, preferably ketamine·HCl.

Ketamine is understood to mean (S)-(±)-2-(2-chlorophenyl)-2-(methylamino)cyclohexan-1-one, (R)-(±)-2-(2-chlorophenyl)-2-(methylamino)cyclohexan-1-one, and the racemate (RS)-(±)-2-(2-chlorophenyl)-2-(methylamino)cyclohexan-1-one.

(S)-ketamine or a pharmaceutically acceptable salt thereof, especially (5)-ketamine·HCl, is especially preferably present as a single stereoisomer of ketamine, since the analgesic and anaesthetic potency of (S)-ketamine is approximately three times higher than that of the (R) form.

The active ingredient content in the at least one layer can vary within relatively wide limits. A range of from 10 to 65 wt. %, in relation to the dry weight of the layer, can be stated as suitable. In one embodiment, the proportion of active ingredient in the layer lies rather in the lower range, for example if the active ingredient has a strong unpleasant taste, which has to be compensated for by a greater amount of taste-masking agents. In this case, a range of from 10 to 30 wt. % can be stated as suitable active ingredient fraction. In another embodiment, the proportion of active ingredient in the dosage form according to the invention lies rather in the upper range, wherein a content of from 30 to 65 wt. % can be stated as being especially preferred.

(S)-ketamine or a pharmaceutically acceptable salt thereof is especially preferably present in the relevant layer in an amount of from 40 to 60 wt. % in relation to the dry weight of the layer in question.

The multi-layer oral thin film according to the invention is also preferably characterised in that at least one layer of the multi-layer oral thin film comprises at least one auxiliary substance selected from the group comprising colouring agents, flavourings, sweeteners, plasticisers, taste-masking agents, emulsifiers, enhancers, humectants, preservatives and/or antioxidants.

Each of these auxiliary substances is preferably contained in this layer in an amount of from 0.1 to 40 wt. %, preferably from 0.1 to 30 wt. %, especially preferably from 0.1 to 15 wt. %, very especially preferably from 0.1 to 10 wt. %, or 0.1 to 5 wt. %, in relation to the total weight of the layer in question.

Each of the layers can be a smooth film in one embodiment.

Each of the layers can be a porous film, for example in the form of a foam, in another embodiment.

The multi-layer oral thin film according to the invention is preferably characterised in that at least one layer is present in the form of a solidified foam that has voids.

The voids and the associated larger surface area of the films facilitate especially the access of water or saliva or other bodily fluids into the interior of the dosage form and thus accelerate the dissolution of the dosage form and the release of the active ingredient.

In the case of a rapidly absorbing active ingredient, the transmucosal absorption can also be improved by the rapid dissolution of the relevant layer.

On the other hand, the wall thickness of said voids is preferably small, as these represent solidified bubbles, for example, so that rapid dissolution or destruction of these voids takes place.

A further advantage of this embodiment is that, despite the comparatively high area density, faster drying can be achieved by formulating as a foam than with a comparable non-foamed composition.

The multi-layer oral thin film according to the invention is preferably characterised in that the voids are isolated from one another and are preferably present in the form of bubbles, the voids being filled with air or a gas, preferably with an inert gas, especially preferably with nitrogen, carbon dioxide, helium or a mixture of at least two of these gases.

According to another embodiment, it is provided that the voids are connected to one another, preferably by forming a continuous channel system penetrating the matrix.

Said voids account preferably have a volume fraction of from 5 to 98%, preferably from 50 to 80%, in relation to the total volume of the layer in question. In this way, the advantageous effect of accelerating the dissolution of the relevant layer is influenced favourably.

Furthermore, surface-active substances or surfactants can be added to the at least one polymer for foam formation or to the obtained foam before or after the drying in order to improve the stability of the foam before or after the drying.

Another parameter that influences the properties of the dosage form according to the invention is the diameter of the voids or bubbles. The bubbles or voids are preferably created with the aid of a foam whipping machine, with which the diameter of the bubbles can be adjusted in a wide range, almost arbitrarily. For example, the diameter of the bubbles or voids may lie in the range of 0.1 to 300 μm. Especially preferably, the diameter is in the range of 50 and 200 μm.

The multi-layer oral thin film according to the invention preferably has an area of from 0.5 cm² to 10 cm², especially preferably from 1.5 cm² to 7.5 cm².

The multi-layer oral thin film according to the invention is preferably characterised in that the area density of each layer of the multi-layer oral thin film is 10 to 500 g/m², preferably 100 to 400 g/m².

The area density of each layer is preferably at least 10 g/m², more preferably at least 20 g/m², or at least 30 g/m², or most preferably at least 50 g/m², or less than or equal to 400 g/m², more preferably less than or equal to 350 g/m², or less than or equal to 300 g/m², or most preferably less than 150 g/m². Preferably, the area density is 10 to 400 g/m², more preferably 20 to 350 g/m 2, or 30 to 300 g/m², and most preferably 50 to 150 g/m².

Preferably, each layer has a layer thickness of from about 20 μm to about 500 μm, especially preferably from about 50 μm to about 300 μm.

The multi-layer oral thin film according to the invention is, in principle, not limited in the number of layers contained.

For example, embodiments are conceivable in which the multi-layer oral thin film has a plurality of active-ingredient-containing layers, which can each contain the same or a different active ingredient.

The at least two layers of the multi-layer oral thin film are preferably not sewn together over their entire surface area.

A sewing not provided over the entire surface area is understood to mean that the area by which the at least two layers of the multi-layer oral thin film are connected to one another is smaller than the surface of one side of a layer of the multi-layer oral thin film. This sewing not provided over the entire surface area can be realised in the form of one or more continuous strips over the surface of the multi-layer oral thin film or by sewing at one or more separate points.

It is preferred that at least one of the overlapping edges of the at least two layers arranged one on top of the other is not closed by a stitching. This has the advantage that saliva can easily penetrate from outside between the individual layers, which improves the dissolution time of the multi-layer oral thin film according to the invention.

The at least two layers of the multi-layer oral thin film according to the invention preferably have the same size or surface area.

If the at least two layers of the multi-layer oral thin film according to the invention have the same size or surface area, it is thus preferred that the at least two layers are arranged congruently one on top of the other, so that the edges of the at least two layers overlap and neither of the at least two layers protrudes beyond the other.

However, embodiments of the oral thin film according to the invention in which the at least two layers are shifted relative to one another so that they are not arranged congruently one on top of the other are also conceivable.

Embodiments of the oral thin film according to the invention in which the at least two layers have a different size or surface area are additional conceivable.

The multi-layer oral thin film according to the invention is preferably characterised in that the at least one thread comprises or consists of at least one water-soluble polymer.

The multi-layer oral thin film according to the invention is preferably characterised in that the at least one thread comprises or consists of at least one polymer which is selected from the group comprising starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and/or natural gums.

A thread comprising or consisting of such polymers has the advantage that it dissolves fully in the patient's mouth and therefore does not have to be removed.

The at least one thread preferably comprises or consists of the same polymer that is also contained in one of the layers of the oral thin film.

However, embodiments in which the at least one thread comprises or consists of a different polymer that the layers of the multi-layer oral thin film are also conceivable.

The multi-layer oral thin film according to the invention is preferably characterised in that the at least one thread has a thickness of from 0.05 to 1 mm, preferably from 0.1 to 0.5 mm, especially preferably of approximately 0.2 mm.

The multi-layer oral thin film according to the invention is preferably characterised in that the at least one thread has a different colour than at least one of the at least two layers, preferably a different colour than all layers.

This has the advantage that the thread can be used to embroider markers into the multi-layer oral thin film. These markers can also be realised independently of the stitching of the individual layers, preferably in the form of an embroidering.

Besides the stitching, a further thread is preferably used, by means of which the multi-layer oral thin film is embroidered and thus marked on at least one layer.

Information, such as the active ingredient contained, the dosage, a brand name, or other information are conceivable as a marking.

The present invention also relates to a method for producing a multi-layer oral thin film as described above comprising the steps of:
a) providing at least one first and one second layer of the multi-layer oral thin film, each comprising at least one polymer;
b) arranging these at least two layers one on top of the other; and
c) connecting these at least two layers by sewing using at least one thread.

The at least two layers are sewn preferably by hand using a suitable needle and a suitable thread.

In another embodiment the sewing is performed using a suitable sewing machine.

The present invention additionally relates to a multi-layer oral thin film, as described above or obtainable by the method described above, wherein especially ketamine, preferably S-ketamine, is used as active pharmaceutical ingredient, for use as a medicament, especially in the treatment of pain and/or depression, especially to reduce the risk of suicide and/or for use as a general anaesthetic, preferably to initiate and carry out general anaesthesia, or as a supplement in the case of local anaesthesia and/or as an analgesic.

The preferred embodiments described above for the multi-layer oral thin film according to the invention are also applicable for the method according to the invention, the multi-layer oral thin film obtained by this method, and use thereof as a medicament.

The invention will be explained in greater detail hereinafter on the basis of non-limiting examples.

### EXAMPLES

### Example 1

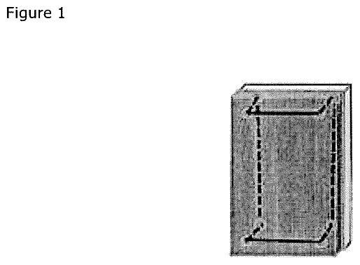

An example of a suitable sewing of two layers in an oral thin film is shown in FIG. 1. Further examples with sewing not provided over the entire surface area, different stitching patterns, and markings by coloured threads are conceivable.

### Example 2

Two foamed layers were produced for producing a multi-layer OTF with the composition according to Table 1 and Table 2.

Table 1 states the composition of a layer of the oral thin film comprising the active pharmaceutical ingredient esketamine (S-ketamine).

Table 2 states the composition of a layer of the oral thin film containing a relatively high amount of tris buffer. This layer is used in the administration of the finished oral thin film in the patient's mouth to establish a pH value that is suitable or advantageous for the absorption of the active pharmaceutical ingredient.

TABLE 1
Active-ingredient-containing layer (size 2.72 cm²):

Theoretical amount

| | per dose | percentage |
|---|---|---|
| Ingredients | [mg/2.72 cm²] | [%] |
| Active ingredient: | | |
| Esketamine HCl | 16.1 | 50.00 |
| Auxiliary substances: | | |
| Polyvinyl alcohol 4-88 | 12.8 | 39.50 |
| Saccharin Na | 0.3 | 1.00 |
| Sucralose | 0.6 | 2.00 |
| Glycerol | 1.5 | 4.50 |
| Masking flavour PHL-131985 | 0.3 | 1.00 |
| Raspberry flavour PHL-097336 | 0.6 | 2.00 |
| Total¹ | 32.3 | 100.00 |
| Other constituents: | | |
| Purified water² | residual | residual |

¹Sum of the non-rounded individual values
²Purified water serves as a process solvent in the production process and is for the most part evaporated during the drying process. Expected value: 2.6%.

In the foam OTF, nitrogen is used as processing aid for foaming.

TABLE 2
Buffer layer (size 2.72 cm²):

Theoretical amount

| | per dose | percentage |
|---|---|---|
| Ingredients | [mg/2.72 cm²] | [%] |
| Buffer: | | |
| Trometamol (TRIS) | 10.5 | 20.00 |
| Auxiliary substances: | | |
| Polyvinyl alcohol 4-88 | 19.0 | 36.15 |
| Polyvinyl alcohol 40-88 | 19.0 | 36.15 |
| Saccharin Na | 0.5 | 1.00 |
| Sucralose | 1.0 | 2.00 |
| Glycerol | 2.4 | 4.50 |
| FD&C Red 40 (Allura Red AC) | 0.1 | 0.20 |
| Total¹ | 52.4 | 100.0 |
| Other constituents: | | |
| Purified water² | residual | residual |

¹Sum of the non-rounded individual values
²Purified water serves as a process solvent in the production process and is for the most part evaporated during the drying process. Expected value: 3.9%

In the foam OTF, nitrogen is used as processing aid for foaming.

The individual layers were produced by mass production techniques known to a person skilled in the art, for example by stirring/mixing the contained components by means of a stirring motor and suitable stirring tools. Gas is injected into the resultant mass by stirring, for example by means of a foam whipping machine. The foamy mass is coated onto a coating substrate in a constant layer thickness using suitable equipment (roller applicator, squeegee, coating box, etc.). All temperature-stable web-like materials from which the dry film can be removed again can serve as coating substrates. This can be ensured by the material selection of the coating substrate (different surface tensions between foamy mass and substrate), or by suitable dehesive coatings of the coating substrate with, for example, silicones or fluoropolymers. The process solvent or solvents contained, usually water or mixtures of water and organic, water-miscible solvents, are removed by drying. The oral thin films can be provided in the appropriate size by cutting or punching from the solid foam layer thus obtained.

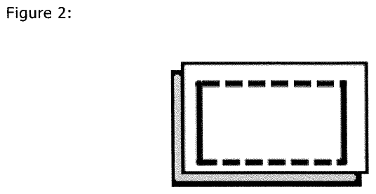

The two layers (active ingredient layer and buffer layer) were placed one on top of the other and connected by sewing using a PVA filament 0.20 mm (Vis Extrusion GmbH, Hohberg, Germany). The two layers placed one on top of the other and also the stitching pattern connecting the two layers are shown schematically in FIG. 2.

The two-layer oral thin film produced in this way was subjected to a stability study, in which it was examined how much active pharmaceutical ingredient migrated into the buffer layer after storage, or the pH value in the active-ingredient layer was determined.

Stability study (3 or 6 weeks' storage at 40° C.)

| pH value unstored | pH value after 3 weeks | pH value after 6 weeks |
|---|---|---|
| 5.75 | 6.46 | 6.48 |

n = 3

| esketamine in buffer | esketamine in buffer | esketamine in buffer |
|---|---|---|
| layer | layer | layer |
| unstored | after 3 weeks | after 6 weeks |
| — | 1 mg | 1 mg |

n = 3

The storage studies show that the migration of substances between the individual layers, when these are connected by a stitched seam, can be minimised.

1. A multi-layer oral thin film, comprising at least two layers arranged one on top of the other which each comprise at least one polymer, characterised in that these at least two layers are connected to one another by at least one stitched seam using at least one thread.

2. The multi-layer oral thin film according to claim 1, characterised in that the at least one polymer in each case comprises a water-soluble polymer.

3. The multi-layer oral thin film according to claim 1, characterised in that the at least one polymer is in each case selected from the group comprising starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, polyvinyl alcohols, polyvinyl alcohol copolymers, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and/or natural gums.

4. The multi-layer oral thin film according to claim 1, characterised in that at least one layer of the multi-layer oral thin film comprises at least one active pharmaceutical ingredient, preferably selected from the group comprising analgesics, hormones, hypnotics, sedatives, antiepiletics, analeptics, psychoneurotropic drugs, neuro-muscle blockers, antspasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active ingredients, antibiotics, chemotherapeutics and narcotics.

5. The multi-layer oral thin film according to claim 1, characterised in that at least one layer of the multi-layer oral thin film comprises ketamine, preferably (S)-ketamine and/or a pharmaceutically acceptable salt thereof.

6. The multi-layer oral thin film according to claim 1, characterised in that at least one layer of the multi-layer oral thin film comprises at least one auxiliary substance selected from the group comprising colouring agents, flavourings, sweeteners, plasticisers, taste-masking agents, emulsifiers, enhancers, humectants, preservatives and/or antioxidants.

7. The multi-layer oral thin film according to claim 1, characterised in that at least one layer of the multi-layer oral thin film is present in the form of a solidified foam that has voids.

8. The multi-layer oral thin film according to claim 7, characterised in that the voids are isolated from one another and are present in the form of bubbles filled with air or a gas or characterised in that the voids are connected to one another and form a channel system penetrating the matrix layer.

9. The multi-layer oral thin film according to claim 7, characterised in that said voids account for a volume fraction of from 5 to 98%, in relation to the total volume of the layer in question.

10. The multi-layer oral thin film according to claim 1, characterised in that the at least one thread comprises or consists of at least one water-soluble polymer.

11. The multi-layer oral thin film according to claim 1, characterised in that the at least one thread comprises or consists of at least one polymer which is selected from the list comprising starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and/or natural gums.

12. The multi-layer oral thin film according to claim 1, characterised in that the at least one thread has a thickness of from 0.05 to 1 mm, preferably of from 0.1 to 0.5 mm.

13. The multi-layer oral thin film according to claim 1, characterised in that the at least one thread has a colour different from at least one of the at least two layers.

14. A method for producing a multi-layer oral thin film according to claim 1, comprising the steps of:
a) providing at least one first and one second layer of the multi-layer oral thin film, each comprising at least one polymer;
b) arranging these at least two layers one on top of the other; and
c) connecting these at least two layers by sewing using at least one thread.

15. (canceled)

16. A method of administrating a medicament comprising providing the multilayer oral thin film of claim 1 to a subject.

17. The multi-layer oral thin film according to claim 7, characterised in that the voids are isolated from one another and are present in the form of bubbles filled with nitrogen, carbon dioxide, helium or a mixture of at least two of these gases.

18. The multi-layer oral thin film according to claim 7, characterised in that the voids are connected to one another and form a channel system penetrating the matrix layer.

19. The multi-layer oral thin film according to claim 7, characterised in that said voids account for a volume fraction of from 50 to 80%, in relation to the total volume of the layer in question.

20. The multi-layer oral thin film according to claim 1, characterised in that the at least one thread has a thickness of from 0.1 to 0.5 mm.
